(19) United States
(12) Patent Application Publication
(10) Pub. No.: US 2024/0042504 A1
(43) Pub. Date: Feb. 8, 2024

(54) METHOD AND DEVICE FOR PRODUCING A TUBULAR SEMI-FINISHED PRODUCT FOR A SCAFFOLD OF AN IMPLANT

(71) Applicant: BIOTRONIK AG, Buelach (CH)

(72) Inventors: ALEXANDER HEUSS, Bad Doberan (DE); ULLRICH BAYER, Bad Doberan (DE); OKECHUKWU ANOPUO, Rostock (DE); ANDRÉ SCHOOF, Bad Doberan (DE); BERND BLOCK, Leonberg (DE); JAN HANNEMANN, Rostock (DE)

(21) Appl. No.: 18/546,643

(22) PCT Filed: Feb. 8, 2022
(86) PCT No.: PCT/EP2022/052951

§ 371 Date: Aug. 16, 2023

(30) Foreign Application Priority Data
Mar. 24, 2021 (EP) 21164643.5

(51) Int. Cl. B21C 23/08; B21C 23/00; B21C 29/00; A61L 31/02

(52) U.S. Cl. CPC B21C 23/085; B21C 23/002; B21C 29/003; A61L 31/022

(57) ABSTRACT

A method for producing a tubular semi-finished product for an implant scaffold which leads to an improvement of the dilatability of the scaffold. The semi-finished product consists of a magnesium alloy. The method includes extruding a tubular semi-finished product by a heated die or tempering an extruded tubular semi-finished product by a heating device. A tube drawing device applies a tensile stress and/or a torsional stress. The tube drawing device has a clamping device. The clamping device is fixed on a predefined portion of the tubular semi-finished product. Tensile force generated by the tube drawing device and/or the torsion moment generated by the tube drawing device is transferred to the semi-finished product.

### PRIORITY CLAIM

This application is a 35 U.S.C. 371 US National Phase and claims priority under 35 U.S.C. § 119, 35 U.S.C. 365(b) and all applicable statutes and treaties from prior PCT Application PCT/EP2022/052951, which was filed Feb. 8, 2022, which application claimed priority from EP Application Number 21164643.5, which was filed Mar. 24, 2021.

### FIELD OF THE INVENTION

The invention relates to a method for producing a tubular semi-finished product for a structural scaffold of a medical implant, wherein the semi-finished product consists of a magnesium alloy and/or a zinc alloy, and to a corresponding device and a semi-finished product produced by the method. The structural scaffold will be referred to hereinafter as a scaffold. The invention also relates to an implant having a structural scaffold in the form of a tubular basic structure.

### BACKGROUND

Bioresorbable scaffolds formed from a magnesium alloy are used in resorbable implants intended for vascular surgery, for example coronary or peripheral implants, for example for stents or in heart valve replacement. The scaffolds are produced by being cut out from a tubular semi-finished product, for example by laser. The scaffolds are often introduced minimally invasively into the body of the patient. For implantation of this kind, the scaffold has a state with a small diameter, in order to transport it, for example along the blood vessels of the patient, to the site of the treatment. There, such a scaffold is then transferred, for example by a balloon, into a state with a larger diameter (expanded, dilated) in order to perform the desired function in the body of the patient, for example a supporting function for a vessel.

Bioresorbable scaffolds formed from magnesium alloys, on account of their hexagonal lattice structure and their limited number of glide planes, have fundamental disadvantages in plastic forming processes, such as dilation. In the past it has therefore been attempted to provide improvements in respect of tubular semi-finished products for the scaffold by way of alloying and process-related measures. In that case, tests leading to an optimised grain size and the most homogeneous distribution possible of intermetallic phases played a key role. This was usually achieved—with a predefined alloy composition—by the optimisation of thermomechanical forming processes.

Semi-finished tubes can be produced in different ways. Known methods, such as tube drawing, rod drawing or also extrusion bring about a preferred orientation of the grains in the microstructure. This anisotropic microstructure or grain structure significantly influences the mechanical properties of the implant scaffolds produced from the semi-finished products. The semi-finished tubes have advantageous mechanical properties in the direction of their longitudinal axis. This circumstance, however, which is favourable for the tube properties, cannot be utilised to the fullest extent in a scaffold manufactured from these tubes, since this scaffold must also withstand stress moments during the dilation process which do not coincide with the deformation direction.

For example, in the known multi-stage tube drawing process with interstage annealing, the limited formability at room temperature caused by the hexagonal, very close-packed magnesium lattice is taken into consideration. For this reason, only small degrees of forming can be achieved per drawing step at room temperature. The deformation capability is improved by intermediate annealing steps, and the semi-finished product in the form of an internally bored blank is deformed until the desired end dimension is achieved. The recurrent deformation capability requires a fully recrystallised microstructure. If this is not achieved after intermediate annealing steps, destruction of the semi-finished product or irreversible crack formation cannot be ruled out. Due to the numerous forming steps and the necessary intermediate annealing steps, this method is very disadvantageous from an economical viewpoint as well.

A single-stage warm extrusion by forward or backward hollow extrusion is also prior art. In such an extrusion process, a bored blank is pressed through a heated die. The thermal influences acting during this process are decisive for the characteristics of the mechanical properties. A high forming temperature causes a complete recrystallisation in the microstructure and thus approximately isotropic material properties. However, in the known method, grain growth occurs, which can reduce the tensile strength and elongation at break. Alternatively, with a low forming temperature the grain growth is prevented or hindered. Due to the low temperature, however, the microstructure is only partly dynamically recrystallised, resulting in a strong texture, which results in highly anisotropic material properties.

### SUMMARY OF THE INVENTION

A method and device of the invention improve the dilatability of an implant scaffold produced from the semi-finished product.

A preferred method according to the invention has the following steps:
a) extruding the tubular semi-finished product by a heated die or tempering the extruded tubular semi-finished product by a heating device,
b) introducing into the material of the semi-finished product exiting from the die or the heating device a tensile stress and/or a torsional stress by a tube drawing device, which has a clamping device, wherein the clamping device is fixed on a predefined portion of the tubular semi-finished product and the tensile force generated by the tube drawing device and/or the torsion moment generated by the tube drawing device transfers to the semi-finished product.

In one exemplary embodiment, the die for extruding the tubular semi-finished product is part of an extrusion device which, besides the die, also includes a ram.

A preferred device according to the invention (tube drawing device) includes a clamping device, wherein the device is designed in such a way that it generates a tensile stress and/or a torsional stress in the material of the semi-finished product, wherein the tensile force generated for this purpose by the device and/or the torsion moment generated by the tube drawing device is transferable by the clamping device to the material of the semi-finished product exiting from the die or the heating device, wherein the clamping device is fixable on a predefined portion of the tubular semi-finished product.

### BRIEF DESCRIPTION OF THE DRAWINGS

FIG. 1 a system according to the invention with a device according to the invention in a view from the side, partly in section,

FIG. 2 a microsection of the microstructure of an extruded tubular semi-finished product formed from a magnesium alloy of magnesium and 6.25% by weight aluminium, which was extruded at 275° C. without the introduction according to the invention of a tensile stress and/or torsional stress,

FIG. 3 a microsection of the microstructure of an extruded tubular semi-finished product formed from a magnesium alloy of magnesium and 6.25% by weight aluminium, which was produced using the system according to the invention at 275° C. and with use of a tensile force of 50 N,

FIG. 4 a comparison of the dilatability (RFD in % above the specification) of implant scaffolds which
a) were produced from extruded tubular semi-finished products from a magnesium alloy, wherein the semi-finished products were produced without the introduction according to the invention of a tensile stress and/or torsional stress, or which
b) were produced from extruded tubular semi0finished products from a magnesium alloy, wherein the semi-finished products were produced using the system according to the invention.

### DETAILED DESCRIPTION OF THE PREFERRED EMBODIMENTS

The material of the tubular semi-finished product includes a magnesium alloy, for example WE43, magnesium-zinc-aluminium, magnesium-aluminium, or magnesium-zinc-calcium. Here, preferably ultra-pure magnesium alloys are used, such as magnesium-zinc-aluminium with 0-4% by weight Zn and 2-10% by weight Al or with 1.5-7% by weight Zn and 0.5-3.5% by weight Al, such as magnesium-aluminium with 5-10% by weight Al, in particular 5.5-7% by weight, particularly preferably 6.25% aluminium, such as magnesium-zinc-calcium with 3-7% by weight Zn and 0.001-0.5% by weight Ca or 0-3% by weight Zn and 0-0.6% by weight Ca. Such ultra-pure magnesium alloys expediently contain, besides the stated alloy elements, less than 0.006% by weight of other elements (impurities such as Fe, Cu, Co, Si etc. or rare earths).

Alternatively or in combination, the material of the tubular semi-finished product includes a zinc alloy, in particular a zinc-magnesium-calcium alloy with 0.2 to 3% by weight Mg, in particular 0.5 to 1.5% by weight Mg, and 0 to 1.5% by weight Ca, in particular 0.01 to 0.5% by weight Ca, wherein the remainder is formed by zinc and unavoidable impurities.

A method according to the invention and the device according to the invention is based on the introduction of additional stresses (tensile and/or torsional stresses) into the tubular semi-finished product. This can be introduced into the tube directly after the tube extrusion process (performed for example at elevated temperatures) or later within the scope of an additional thermally assisted finishing step by the heating device. A metallographic microstructure thus results, which contains grains, of which the preferred orientation does not correspond to the direction of the tube axis and thus the deformation direction of the semi-finished product. The preferred orientation of many grains of the microstructure produced by the method according to the invention runs obliquely or transversely to the longitudinal axis of the tubular semi-finished product, so that a mixed grain orientation is created on the whole. The advantage of this mixed grain orientation then takes effect if scaffolds which are fabricated from these tubes are mechanically loaded and plastically deformed during the dilation process. The scaffold regions that have the greatest plastic expansion during the dilation process are identical to the points of origin of cracks which lead to a later failure of the scaffold. On account of the mixed grain orientation provided with natural torsional stresses, the crack propagation that occurs with ongoing plastic deformation of the scaffold is slowed, so that a scaffold produced from the semi-finished product produced in accordance with the invention fails later on average. Consequently, the dilatability or the plastic deformation capability of an implant scaffold produced from the semi-finished product is improved and larger dilation diameters can be used which are extremely desirable from the viewpoint of clinical safety and in addition significantly increase the safety of this class of medicinal product.

The method according to the invention, which is performed with the device according to the invention, additionally causes a grain refinement. This grain refinement, besides the formation of new grains, also includes the above-described effect in relation to the grain orientation, which in the event of a mechanical load of the scaffold produced from the semi-finished product, advantageously counteracts crack propagation. The solution according to the invention is tailored to the situation of plastic deformation, which is accompanied by crack propagation, wherein the grains of the semi-finished product according to the invention (and thus also of the scaffold produced therefrom) delay crack propagation on account of their orientation. Due to the applied tensile and/or torsional stress, a recrystallisation of amorphous regions of the microstructure is brought about, wherein the grain orientation of the growing grains is different and in particular runs obliquely or transversely to the is longitudinal axis of the tubular semi-finished product. This state is “frozen” in each grain once the recrystallisation is not reached.

A suitable temperature range for the material of the semi-finished product heated in the die and consisting of a magnesium alloy is between 200° C. and 450° C., in particular between 240° C. and 290° C. Alternatively, a suitable temperature range for the tempering of an extruded tubular semi-finished product consisting of a magnesium alloy for preparation for the effect of the tensile and/or torsional stress is between 180° C. and 270° C.

A suitable temperature range for the material of the semi-finished product heated in the die and consisting of a zinc alloy is between 140° C. and 310° C., in particular between 150° C. and 250° C. Alternatively, a suitable temperature range for the tempering of an extruded tubular semi-finished product consisting of a magnesium alloy for preparation for the effect of the tensile and/or torsional stress is between 250° C. and 450° C.

For generation of the tensile force, the tube drawing device has, for example, a slide (puller) which is movable on or in a guide and which has a clamping device fastened thereto, wherein the guide of the slide is arranged on a corresponding holding device or a corresponding stand. The guide can be formed, for example, as a dovetail guide. To move the slide in the guide, a stepper motor or a pneumatic drive is provided, for example. The movement of the slide generates the tensile force, which is transferred to the material of the semi-finished product via the clamping device connected to the slide. If the slide is driven pneumatically, for example, a force/distance diagram can be specified and implemented by the use of electronic proportional valves.

The clamping device can additionally be mounted rotatably and likewise provided with a drive, in particular a pneumatic drive, wherein the longitudinal axis of the rotation runs parallel to the longitudinal axis of the tubular semi-finished produced held by the clamping device. The clamping device transfers a torsion moment to the semi-finished product, wherein the torsional stress can be adjusted as a function of the tube length.

The clamping device in the starting state is preferably arranged a few tenths of a millimetre to 5 mm above the die and engages the semi-finished product with parallel grippers or also with a centric gripper. The distance of the gripper from the die increases over the tube length. The clamping diameter of the grippers can be adjusted, for example by a millimetre screw.

The above object is also achieved by a system including an above-mentioned device (tube drawing device) and
an extrusion device with a heated die for extruding the tubular semi-finished product or
a heating device for tempering an extruded tubular semi-finished product.

The temperature ranges for the die of the extrusion device or the heating device that are advantageous for a magnesium alloy or zinc alloy have already been stated above.

The above object is also achieved correspondingly by a tubular semi-finished product for an implant scaffold produced by the above-mentioned method and by an implant scaffold produced by cutting from the tubular semi-finished product, for example by laser cutting. The cut-out scaffold can then be electropolished and coated as necessary.

The above object is likewise achieved by an implant for implantation in a bodily lumen, wherein the implant includes a tubular basic structure which contains a magnesium alloy and/or zinc alloy, wherein the magnesium and/or zinc alloy has a grain structure formed from uniform and equally distributed grains with a mixed grain orientation, wherein the tubular basic structure in a starting state has a plurality of bars which are oriented at least in part in the circumferential direction. The implant can be compressed by a plastic deformation and expanded by a later plastic deformation to up to 150% of the diameter in the starting state, without any of the bars oriented at least in part in the circumferential direction breaking.

A bar oriented at least in part in the circumferential direction is understood within the scope of the application to mean a bar that has an angle of <90°, in particular <500 with the circumferential direction. A bar that has an angle of 0° with the circumferential direction is oriented accordingly in the circumferential direction. A bar, often also referred to as a strut, is understood to mean an elongate structure that is straight or wound, in particular wound in an S shape. The orientation of a structure wound in this way corresponds to the centre of gravity vector.

An implant for implantation in a bodily lumen is in particular a stent for implantation in a blood vessel.

The starting state is understood to mean the implant as it is obtained after having been cut out from the tubular semi-finished product and after optional electropolishing and/or coating. The starting state of the implant is thus the state before the implant is compressed onto a catheter, in particular a balloon catheter, for insertion of the implant. The compression and thus fastening of the implant on the catheter is often also referred to as crimping.

A breakage of the bars is understood within the scope of the application to mean a crack that passes at least once through the entire cross section of the bars.

The grains in one embodiment have a mean grain size of at most 15 micrometres, preferably at most 10 micrometres.

The invention will be explained hereinafter on the basis of an exemplary embodiment and with reference to the figures. All features that are described and/or shown in the figures, individually or in any combination, form the subject matter of the invention, also independently of their compilation in the claims or the dependency references of the claims.

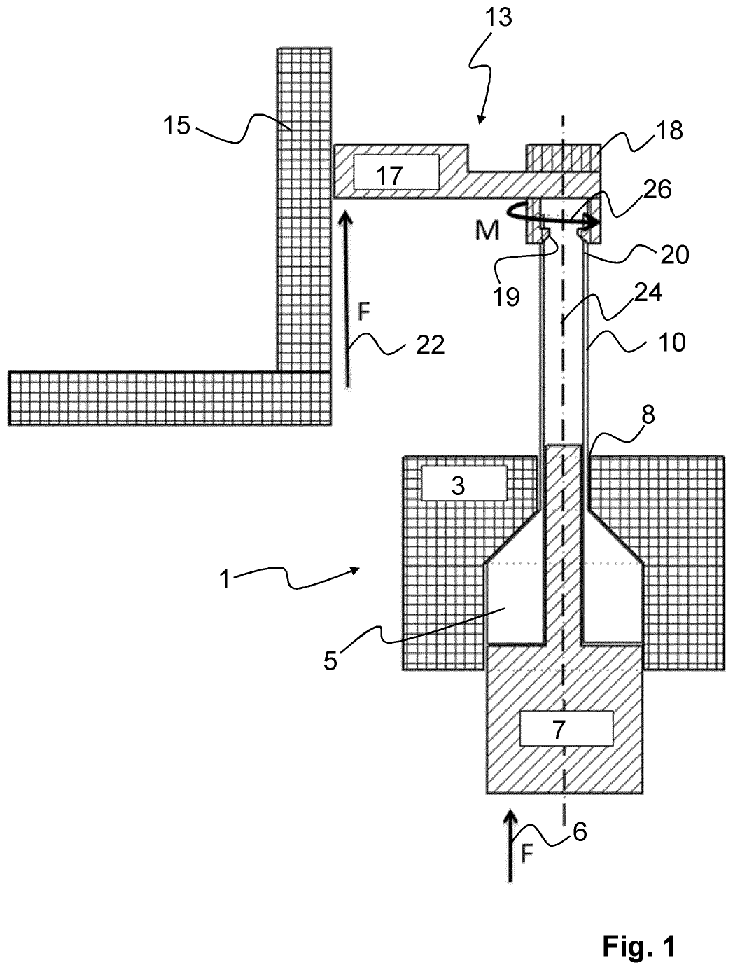

The system according to the invention shown in FIG. 1 is intended for producing a tubular semi-finished product for a scaffold of an implant. The system has an extrusion device 1 with a heated die 3. The material 5 to be extruded is arranged in a recess of the die. The material 5 is pressed from a shaping outlet opening 8 using a ram 7. The outlet opening 8, which is created from the interaction of the ram 7 and the die 3, has a hollow-cylindrical form, so that a tubular or hollow-cylindrical semi-finished product 10 is created. The die 3 heats the material 5, for example in a temperature range between 240° C. and 450° C.

FIG. 1 also shows a tube drawing device 13 with an L-shaped holder 15 and a slide (puller) 17 guided in a rail of the holder 15. A rotary head 18 with a pneumatic drive (not shown) is arranged on an arm protruding from the slide 17, which drive is connected to a clamping device 19 so that the slide 17 is also connected to the clamping device 19. At its end opposite the die 3 of the extrusion device 1, the semi-finished product 10 that has exited from the outlet opening 8 is engaged by the clamping device 19, which is fixedly connected to the semi-finished product 10 in this portion 20.

The slide 17 is moved upwardly (see arrow 22) in the view of FIG. 1 in the rail of the holder 15, for example by a pneumatic drive. The slide 17 generates a tensile force F (see arrow 22), which is transferred via the clamping device 19 to the semi-finished product 10 and brings about a tensile stress in the material of the semi-finished product 10. The pneumatic drive for the slide 17 can include electronic proportional valves, by which a force/distance diagram can be predefined and implemented.

Alternatively or additionally, the clamping device 19 held rotatably in the arm of the slide 17 is rotated by the rotary head 18 by a further pneumatic drive (not shown) about a longitudinal axis which runs parallel to the longitudinal axis 24 of the tubular semi-finished product 10 (for example coincides therewith). This is shown in FIG. 1 by the arrow 26. A torsion moment M is hereby generated, which is transferred via the clamping device 19 to the semi-finished product 10 and brings about a torsional stress in the material of the semi-finished product 10. The torsion moment is adjusted as a function of the length of the semi-finished product 10.

It should be emphasised at this juncture that, by the tube drawing device 13 according to the invention acting on the semi-finished product 10, a tensile stress or a torsional stress or both stresses can be generated in the material of the semi-finished product 10 exiting from the extrusion device 1. The tensile force or the torsion moment act here directly on the semi-finished product exiting from the extrusion device.

The clamping device 19, in the starting state, is arranged on the semi-finished product at a distance from the outlet opening 8, wherein the distance is at least 0.2 mm.

Alternatively, an extruded and initially cooled tubular semi-finished product can be tempered by a heating device in a temperature range between 180° C. and 270° C. and a tensile stress and/or torsional stress is then introduced into the heated material of the semi-finished product by the above-described pipe drawing device 13.

The tensile stress introduced into the material of the semi-finished product 10 lies in the range of from 20 N/mm² to 100 N/mm², preferably 40 N/mm², and the introduced torsional stress lies in the range of 0-100% of the introduced tensile stress, preferably 50% of the tensile stress.

The semi-finished product 10 provided with a tensile stress and/or a torsional stress is then cooled in both embodiments.

The implant according to the invention, for example a stent, can be produced from the semi-finished product 10 in the known manner by laser cutting and subsequent electropolishing.

The material of the semi-finished product 10 and thus of the scaffold or the implant is in this embodiment a magnesium alloy, for example WE43, magnesium-zinc-aluminium, magnesium-aluminium or magnesium-zinc-calcium.

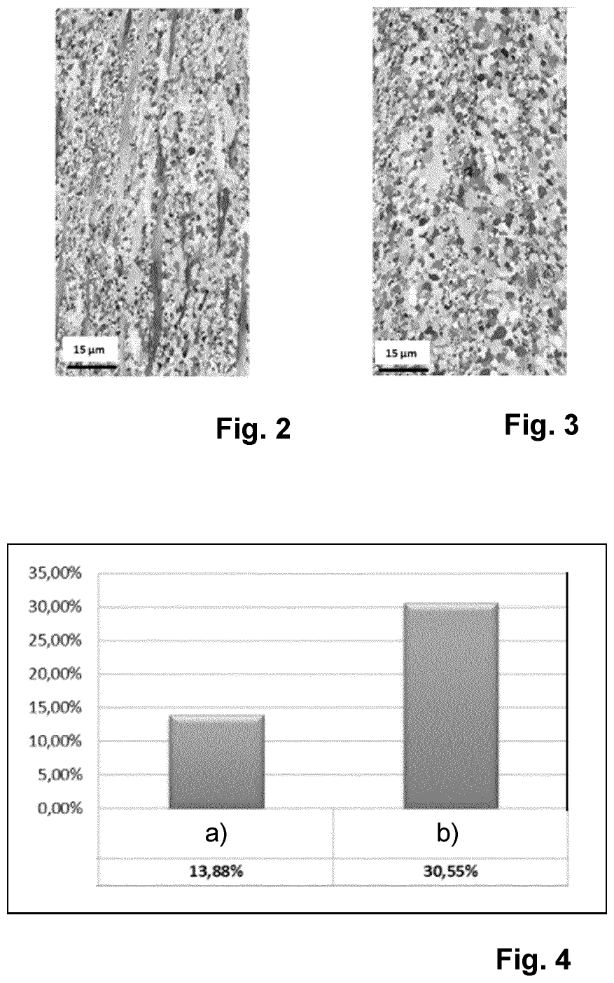

As has already been explained above, the introduced stresses cause a grain refinement, with the adjacent grains of the microstructure having a slightly different orientation. The grains are therefore no longer—as in the conventional methods—oriented only in the preferred direction along the longitudinal axis of the tubular semi-finished product (see FIG. 2), but are smaller and have a mixed orientation. This can be seen very well in FIG. 3, which shows the microstructure once tensile and/or torsional stresses have been introduced by the tube drawing device 13 according to the invention. More sliding planes are thus available—in the event of plastic deformation, stress peaks in a grain are removed again by the next grain. In addition, the speed of growth of cracks reduces as a result of the torsion moments contained in the material, since cracks in the grains are forced to change their direction. The introduced tensile and/or torsional stresses additionally ensure an increased recrystallisation rate (dynamic recrystallisation). Due to the stresses, the nucleation during the forming is facilitated. A greater number of new grains is thus created, whereby the proportion of the recrystallised microstructure increases (see FIG. 3). As a result of this process, isotropic material properties are promoted without grain growth as a result of thermomechanical process influencing. In addition, the microstructure homogeneity in the overall tubular semi-finished product 10 is increased, such that a uniform texture is produced along the entire tube length and the entire tube cross section also in the circumferential direction. This in turn increased the isotropic response of a scaffold produced from the semi-finished product during the crimping or the dilation as a prerequisite for use of the scaffold as an implant in the treated bodily lumen.

The improvement of the material properties is evident from the graph shown in FIG. 4. By measuring what is known as the “rated fracture diameter (RFD)”, i.e. the diameter of the scaffold obtainable with statistic certainty during dilation until breakage, it is possible to assess the dilatability. FIG. 4 shows a comparison of the RFD for scaffolds produced from conventional semi-finished products (a)) and scaffolds whose semi-finished product was produced by the method according to the invention (b)). An increase of the dilatability by more than 16% is determined.

### LIST OF REFERENCE SIGNS

1 extrusion device
3 heated die
5 material to be extruded
7 ram
8 outlet opening
10 tubular semi-finished product
13 tube drawing device
15 holder
17 slide (puller)
18 rotary head
19 clamping device
20 portion of the semi-finished product 10
22 arrow
24 longitudinal axis of the semi-finished product 10
26 arrow

1. A method for producing a tubular semi-finished product for a scaffold of an implant, wherein the semi-finished product consists of a magnesium alloy and/or a zinc alloy, said method comprising:
extruding a tubular semi-finished product in a heated die or tempering an extruded tubular semi-finished product in a heating device,
applying a tensile stress and/or a torsional stress to the tubular semi-finished product or the extruded tubular semi-finished product by a tube drawing device, the tube drawing device comprising a clamping device, the clamping device being fixed on a predefined portion of the tubular semi-finished product or the extruded tubular semi-finished product, wherein tensile force and/or the torsion moment generated by the tube drawing device transfers to the tubular semi-finished product or the extruded tubular semi-finished product.

2. The method according to claim 1, wherein the die applies heat to material of the semi-finished product in a range between 200° C. and 450° C.

3. The method according to claim 1, wherein the heating device applies heat to the extruded tubular semi-finished product in a range between 180° C. and 270° C.

4. The method according to claim 1, wherein a slide of the tube drawing device is movable on a guide, the clamping device is connected to the slide, and the slide applies the tensile stress.

5. The method according to claim 1, wherein a rotary head of the tube drawing device is rotatable about a longitudinal axis that runs parallel to a longitudinal axis of the tubular semi-finished product and the clamping device is connected to the rotary head.

6. A device for producing a tubular semi-finished product for a scaffold of an implant for use with a heated die or a heating device, the device comprising a clamping device configured to apply a tensile stress and/or a torsional stress in the material of the semi-finished product exiting from the die or the heating device, wherein the clamping device is configured to be fixed on a predefined portion of the tubular semi-finished product.

7. The device according to claim 6, comprising a slide movable on or in a guide, the slide being connected to the clamping device and being configured to apply the tensile stress.

8. The device according to claim 7, comprising a rotary head that is rotatable about a longitudinal axis, the rotary heading being connected to the clamping devices, wherein a longitudinal axis of the rotation of the rotary head runs parallel to a longitudinal axis of the tubular semi-finished product, and the rotary head is configured to apply the torsional stress.

9. A system comprising a device according to claim 6, comprising
an extrusion device with a heated die for extruding the tubular semi-finished product or
a heating device for tempering an extruded tubular semi-finished product.

10. The system according to claim 9, the heated die is configured to apply a temperature between 200° C. and 450° C.

11. The system according to claim 9, wherein the heating device is configured to apply a temperature between 180° C. and 270° C.

12. (canceled)

13. (canceled)

14. An implant for implantation in a bodily lumen, comprising a scaffold in the form of a tubular basic structure which comprises a magnesium alloy and/or zinc alloy, wherein the magnesium and/or zinc alloy has a grain structure formed from uniform and equally distributed grains with a mixed grain orientation, wherein the tubular basic structure in a starting state has a plurality of bars which are oriented at least in part in the circumferential direction, wherein the implant is compressed by a plastic deformation and can be expanded by a later plastic deformation to up to 150% of the diameter in the starting state, without any bars oriented at least in part in the circumferential direction breaking.

15. The implant according to claim 14, wherein the grains have a mean grain size of at most 15 μm.

16. The implant according to claim 15, wherein the grains have a mean grain size of at most 10 μm.
